(19) United States
(12) Patent Application Publication
(10) Pub. No.: US 2026/0355509 A1
(43) Pub. Date: Nov. 12, 2026

(54) BIMETAL DIFFUSION BONDED ELECTRODE AND METHODS OF USING THE SAME

(71) Applicant: Medtronic, Inc., Minneapolis, MN (US)

(72) Inventors: Bernard Q. Li, Plymouth, MN (US); Richard T. Stone, Roseville, MN (US); Dale F. Seeley, Spring Park, MN (US)

(21) Appl. No.: 19/125,066

(22) PCT Filed: Oct. 12, 2023
(86) PCT No.: PCT/IB2023/060294

§ 371 Date: Apr. 28, 2025

Related U.S. Application Data
(60) Provisional application No. 63/420,430, filed on Oct. 28, 2022.

(51) Int. Cl. A61N 1/05; B23K 20/02; B23K 101/36; B23K 103/18; C22C 5/04; C22C 14/00

(52) U.S. Cl. CPC A61N 1/0551; B23K 20/02; C22C 5/04; C22C 14/00; B23K 2101/36; B23K 2103/18

(57) ABSTRACT

An electrode according to the present disclosure includes an inner metal layer; and an outer metal layer disposed at least partially around and diffusion bonded to the inner metal layer. The inner metal layer may include Titanium or a Titanium alloy, and the outer metal layer may include Platinum or a Platinum alloy.

### BACKGROUND

The present disclosure is generally directed to electrodes, and relates more particularly to electrodes formed with diffusion bonding.

Electrical leads may be used for a variety of purposes. In the medical industry, electrical leads may be used to deliver current to an anatomical element, such as a nerve. Some electrical leads may be radiopaque, allowing them to appear on fluoroscopic images. Metal alloys are popular material for use in the construction of electrical leads.

### BRIEF SUMMARY

Example aspects of the present disclosure include:

An electrode according to at least one embodiment of the present disclosure comprises: an inner metal layer; and an outer metal layer disposed at least partially around and diffusion bonded to the inner metal layer.

Any of the features herein, wherein the inner metal layer comprises Titanium or a Titanium alloy.

Any of the features herein, wherein the Titanium alloy comprises Titanium and one or more of Tantalum, Niobium, Molybdenum, Zirconium, Tin, Vanadium, Tungsten, Iron, and Aluminum.

Any of the features herein, wherein the inner metal layer is a Titanium-15Molybdenum alloy.

Any of the features herein, wherein the outer metal layer comprises Platinum or a Platinum alloy.

Any of the features herein, wherein the Platinum alloy comprises Platinum and Iridium.

Any of the features herein, wherein the diffusion bonding is performed at a temperature of between 730 degrees Celsius (°C) and 750 °C and for a time between 175 minutes and 300 minutes.

Any of the features herein, wherein the diffusion bonding is performed at a temperature of between 730 °C and 738 °C and for a time of between 175 minutes and 200 minutes.

Any of the features herein, wherein the diffusion bonding occurs in a high pressure environment that includes an inert gas.

Any of the features herein, wherein the inert gas comprises argon, and wherein a pressure of the high pressure environment is about 30,000 pound-force per square inch (psi).

A method according to at least one embodiment of the present disclosure comprises: extruding an inner metal layer and an outer metal layer at least partially disposed around the inner metal layer; and diffusion bonding the inner metal layer and the outer metal layer.

Any of the features herein, wherein the diffusion bonding is performed at a temperature of between 730 degrees Celsius (°C) and 750 °C and for a time between 175 minutes and 300 minutes.

Any of the features herein, wherein the diffusion bonding is performed at a temperature of between 730 °C and 738 °C and for a time of between 175 minutes and 200 minutes.

Any of the features herein, wherein the inner metal layer comprises Titanium or a Titanium alloy.

Any of the features herein, wherein the Titanium alloy comprises Titanium and one or more of Tantalum, Niobium, Molybdenum, Zirconium, Tin, Vanadium, Tungsten, Iron, and Aluminum.

Any of the features herein, wherein the inner metal layer is a Titanium-15Molybdenum alloy.

Any of the features herein, wherein the outer metal layer comprises Platinum or a Platinum alloy.

Any of the features herein, wherein the Platinum alloy comprises Platinum and Iridium.

A system according to at least one embodiment of the present disclosure comprises: an implantable neural stimulator; an electrode electrically connected to the implantable neural stimulator, the electrode comprising: an inner metal layer; and an outer metal layer disposed at least partially around and diffusion bonded to the inner metal layer; a processor; and a memory including data stored thereon that, when processed by the processor, cause the processor to: receive information associated with a first electrical pulse; and causing the implantable neural stimulator to send the first electrical pulse to the electrode.

Any of the features herein, wherein the inner metal layer comprises Titanium or a Titanium alloy, and wherein the outer metal layer comprises Platinum or a Platinum alloy.

Any of the features herein, wherein a Titanium-15Molybdenum (Ti-15Mo) wire connects the electrode to the implantable neural stimulator, and wherein the Ti-15Mo wire has twice a strain capability for a first fatigue life as compared to a 35% Cobalt, 35% Nickel, 20% Chromium, and 10% Molybdenum alloy.

A system according to at least one embodiment of the present disclosure comprises: an implantable neural stimulator; a lead with a proximal end connectable to the implant neural stimulator and a distal end that includes at least one electrode, the at least one electrode comprising: an inner metal layer; and an outer metal layer disposed at least partially around and diffusion bonded to the inner metal layer.

Any of the features herein, wherein the lead comprises a Titanium-15Molybdenum (Ti-15Mo) wire connects the at least one electrode to connector rings attached to the proximal end of the lead.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1A is a conceptual diagram of a system according to at least one embodiment of the present disclosure;

FIG. 1B is a block diagram of aspects of a system according to at least one embodiment of the present disclosure;

FIG. 2 is a diagram of aspects of the system according to at least one embodiment of the present disclosure;

FIG. 3A is a diagram of an electrode according to at least one embodiment of the present disclosure;

FIG. 3B is a diagram of the electrode connected to a wire according to at least one embodiment of the present disclosure;

FIG. 4 is an illustration of electrodes connected to a straight wire according to at least one embodiment of the present disclosure;

FIG. 5A is an image of the electrode according to at least one embodiment of the present disclosure;

FIG. 5B is another image of the electrode according to at least one embodiment of the present disclosure;

FIG. 5C is an image of a diffusion layer according to at least one embodiment of the present disclosure;

FIG. 5D is another image of a diffusion layer according to at least one embodiment of the present disclosure; and

FIG. 6 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the Implantable Neural Stimulator (INS) and further from stimulating electrodes, and distal being closer to the stimulating electrodes, and further from the INS.

Electrodes may include an 85% Titanium-15% Molybdenum (Ti-15Mo) wire that has approximately twice the strain capability for a given fatigue life compared to, for example, MP35N® (e.g., a 35% Cobalt, 35% Nickel, 20% Chromium, and 10% Molybdenum alloy). For example, the Ti-15Mo wire may have a 1% elastic limit, while the MP 35N® elastic limit may be around 0.4%.

However, the Ti-15Mo wire contains Titanium (Ti), making it difficult to combine the wire with Platinum (Pt) using liquid phase welding processes such as laser welding or resistance welding for the purposes of creating an electrode. The bimetal electrodes are desired to meet certain weldability, electrode charge density, and radiopacity requirements.

In accordance with at least one embodiment of the present disclosure, a bimetal ring may be used in the construction of an electrode. The inner portion of the bimetal ring may include a Titanium alloy that is weldable to the wire (e.g., a Ti-15Mo wire), while the outer portion of the bimetal ring may include Platinum or a Platinum alloy (e.g., a Platinum-Iridium alloy). Such a construction may enable the bimetal ring to be compatible as a high charge density, neuro-stimulating electrode with high radiopacity. The bimetal ring material may be fabricated into a long section and each individual electrode may be fabricated by parting the electrode off from the end of a composite tube that includes a Titanium alloy inner layer and a Platinum outer layer.

In accordance with at least one embodiment of the present disclosure, the composite alloy tube may be fabricated by extruding a Platinum clad Titanium tube to an appropriate dimension. The Platinum and Titanium alloy can then be bonded together (e.g., using diffusion bonding). The diffusion bonding process may occur by placing the extruded Platinum clad Titanium alloy tube into an oven with an argon atmosphere at approximately 30,000 pound-force per square inch (psi) and baking at a high temperature. This high pressure and high temperature process may be referred to as a HIP process. The diffusion bonding process does not necessarily incorporate a liquid phase process to join the two metals together. The diffusion bonding may occur for 30 minutes, 1 hour, 3 hours, 5 hours, or 6 hours at, for example, anywhere between 730 degrees Celsius (C) and 850 °C.

In accordance with at least one embodiment of the present disclosure, the inner layer of the metal ring electrode may include a Titanium-based alloy such as a commercially pure Titanium or a beta Titanium alloy. The beta Titanium alloy may, in addition to Titanium, contain one or more of Tantalum (Ta), Niobium (Nb), Molybdenum (Mo), Zirconium (Zr), Tin (Sn), vanadium (V), Tungsten (W), Iron (Fe), and Aluminum (Al). The outer layer may comprise Platinum (Pt) or a Platinum alloy. The Platinum in the outer layer should be suitable for tissue contacting the electrode material, such as pure Platinum or an alloy with about 90% Platinum and about 10% Iridium (Ir).

In accordance with at least one embodiment of the present disclosure, obstructive sleep apnea (OSA) leads may be configured for high fatigue performance to meet the high flexural cyclic requirements of the human throat. The Titanium alloy lead conductor wire can be used with the OSA lead and can exhibit the very high flexural fatigue capabilities. While the Titanium alloy may not be weldable to Platinum, the composite ring electrode provides a solution for welding the Titanium alloy wire to the inner layer of the electrode while incorporating Platinum on the outer electrode surface to contact patient tissue. The electrode may also demonstrate comparable radiopacity to other electrodes.

Embodiments of the present disclosure provide technical solutions to one or more of the problems of (1) providing high electrode charge density electrodes that are also safe for use inside a patient, (2) connecting Ti-based alloys to Pt-based alloys without experiencing poor bonding, and (3) fabricating electrodes that are radiopaque.

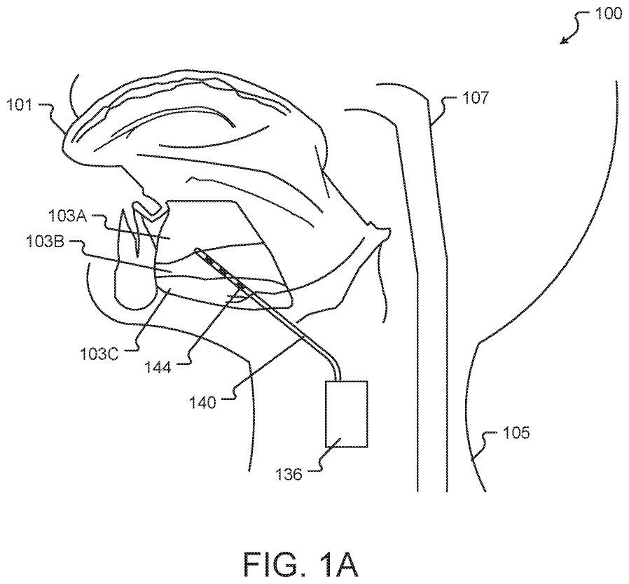

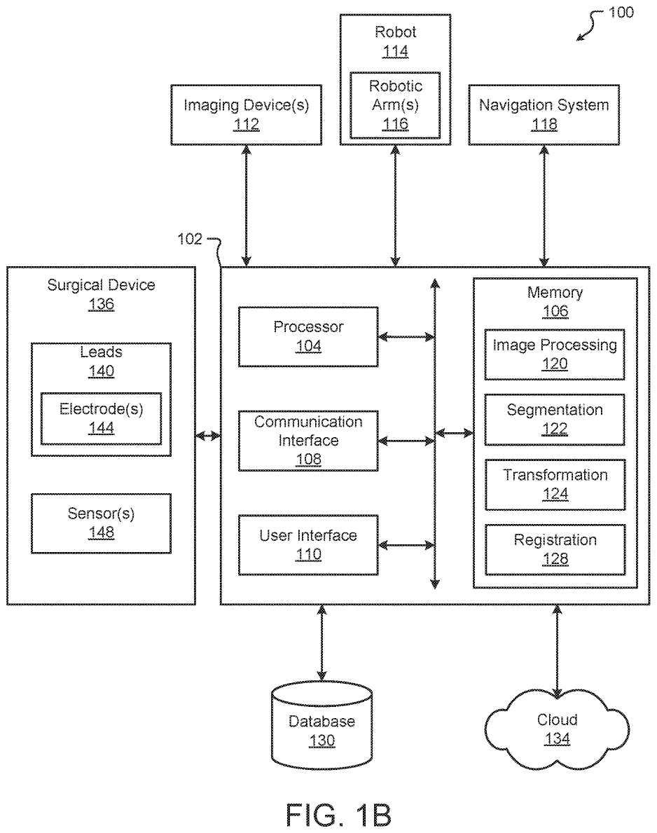

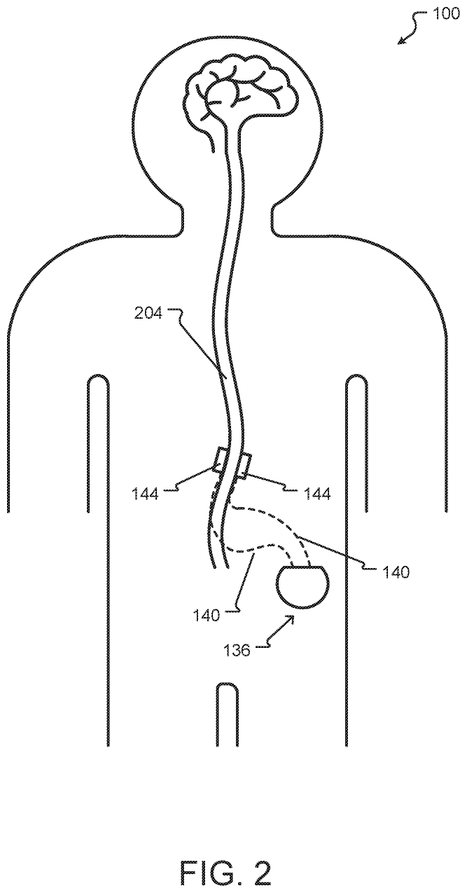

Turning first to FIGS. 1A-1B and 2, a block diagram and other aspects of a system 100 according to at least one embodiment of the present disclosure are shown. The system 100 includes a surgical device 136 (also referred to herein as an internal neural stimulator or an implantable neural stimulator) with one or more leads 140 that is implanted into a patient 105. As discussed below, the lead 140 includes one or more electrodes 144 configured to provide electrical stimulation (e.g., a current) to anatomical tissue. In some embodiments, the surgical device 136 may be used to deliver an OSA therapy. For instance, the lead 140 may be implanted within protrusor muscles 103A-103C of the tongue 101 of the patient 105, such as the genioglossus muscle. The genioglossus muscle includes an oblique compartment 103A and a horizontal compartment 103B, and the lead 140 may be positioned such that the electrodes 144 can stimulate one or both of the oblique compartment 103A and the horizontal compartment 103B.

The OSA therapy delivered by the surgical device 136 (e.g., through stimulation of anatomical tissue using the electrodes 144) may activate the protrusor muscles 103A-103C to move the tongue 101 forward to promote a reduction of obstruction or narrowing of the upper airway 107 during sleep. The activation of the protrusor muscles 103A-103C may be caused by stimulating the protrusor muscles 103A-103C with an electric current delivered by the electrodes 144.

The lead 140 may be implanted using various methods. In some embodiments, a percutaneous procedure may be used to introduce the leads 140 to the patient 105. For example, a surgeon may insert a needle through the lower part of the jaw and tongue 101 of the patient 105 starting from the back of the tongue 101. Once the needle is in place, the surgeon may insert a guidewire through the needle and anchor the guidewire to the tongue 101. Then, the surgeon may place an introducer over the guidewire and, once the introducer is in place, remove the guidewire. The surgeon may then put the lead 140 through the introducer such that the electrodes 144 are positioned within the protrusor muscles 103A-103C. In some embodiments, the surgeon may repeat the above steps for each lead introduced to the patient 105.

The system 100 may additionally or alternatively be used to control, pose, and/or otherwise manipulate an implanted device and/or an implanted electrode, a surgical mount system, a surgical arm, and/or surgical tools attached thereto and/or carry out one or more other aspects of one or more of the methods disclosed herein. The system 100 comprises a computing device 102, one or more imaging devices 112, a robot 114, a navigation system 118, a database 130, a cloud or other network 134, and/or a surgical device 136. Systems according to other embodiments of the present disclosure may comprise more or fewer components than the system 100. For example, the system 100 may not include the imaging device 112, the robot 114, the navigation system 118, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the method 600 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120, segmentation 122, transformation 124, and/or registration 128. Such content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, and/or the cloud 134.

The computing device 102 may also comprise a communication interface 108. The communication interface 108 may be used for receiving image data or other information from an external source (such as the imaging device 112, the robot 114, the navigation system 118, the database 130, the cloud 134, and/or any other system or component not part of the system 100), and/or for transmitting instructions, images, or other information to an external system or device (e.g., another computing device 102, the imaging device 112, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computing device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component.

Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise one or more sensors that enable the processor 104 (or a processor of the robot 114) to determine a precise pose in space of the robotic arm (as well as any object or element held by or secured to the robotic arm).

In some embodiments, reference markers (e.g., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The surgical device 136 (also referred to an Internal Neural Stimulator) may include leads 140, electrodes 144, and one or more sensors 148. The surgical device 136 may be an instrument or implantable device capable of providing electric signals to the electrodes 144 disposed on the distal ends of the leads 140 to cause the electrodes 144 to stimulate proximate patient tissue, one or more anatomical elements (e.g., the spinal cord, one or more nerves, etc.), combinations thereof, and the like. For example, the surgical device 136 may receive instructions from the computing device 102 to generate an electrical pulse capable of providing a stimulation to a nerve 204 of the patient. While nerves and patient tissue are discussed herein, it is to be understood that the electrode may be implanted anywhere within the patient or be disposed on any exterior surface of the patient to stimulate any anatomical tissue or element. As illustrated in FIG. 2, the surgical device 136 may be a device capable of being surgically implanted within the patient. In such embodiments, the surgical device 136 may communicate with the computing device 102 or other components of the system 100 wirelessly. Alternatively, the surgical device 136 may be disposed outside the patient, with the leads 140 and the electrodes 144 being surgically implanted. In some embodiments, the leads 140 may be placed parallel with the spinal column of the patient.

In some embodiments, the electrodes 144 may be disposed on opposite sides of the nerve 204 (e.g., on opposite sides of the spinal cord of the patient). The position and/or orientation of each electrode 144 relative to nerve 204 may vary depending on, for example, the type of treatment, the type of electrode, combinations thereof, and the like. In some examples, the current being applied to each side of the nerve 204 may be different for each electrode 144. For example, a first electrode may apply a high frequency stimulation (e.g., such as a given waveform at about 5 kHz) and a second electrode may apply a low frequency stimulation (e.g., such as a square wave or other waveform at 1Hz) to provide an electrical stimulation signal to the nerve 204.

In some embodiments, the surgical device 136 may include a single electrode 144, while in other embodiments, two or more electrodes 144 may be used. In one embodiment, the electrodes 144 may be disposed proximate muscles in the throat of a patient that control the position of the tongue. Additionally, while not shown, the surgical device 136 may include one or more processors similar to the processor 104 (e.g., one or more DSPs, general purpose microprocessors, graphics processing units, ASICs, FPGAs, or other equivalent integrated or discrete logic circuitry) shown and described in FIG. 1B that are programmed to carry out one or more aspects of the present disclosure. In some examples, the one or more processors may include a memory or may be otherwise configured to perform the aspects of the present disclosure. For example, the one or more processors may provide instructions to the surgical device 136 to perform an electrical stimulation of the nerve 204 by passing a first current through the electrodes 144.

In one embodiment, the surgical device 136 may be or comprise an INS. The INS may be implanted in the patient and attached to the electrodes 144, such that the INS can send an electrical pulse to the electrodes 144 to stimulate patient tissue. Additionally or alternatively, the INS may also use the one or more sensors 148. The sensors 148 may be or comprise micro electrical-mechanical system (MEMS) sensors and/or biological sensors capable of generating information related to one or more measured parameters to enable the INS to sense one or conditions for the purposes of determining whether or not to generate an electrical pulse. For example, the sensors 148 may comprise biosensors that can detect or monitor neurophysiological signals entering or exiting specific organs proximate the INS and/or other parameters associated with the patient (e.g., heart rate, respiration rate, etc.). As another example, the sensors 148 may detect information related to muscles in the throat and send such information to the processor 104. The processor 104 may, based on the information from the sensors 148, determine that the INS should generate and pass an electrical pulse to the electrode 144 to stimulate the muscles to prevent the patient's airway from closing. Alternatively, the electrodes may be used to sense electrical signals within the body tissue to provide for closed loop control of the electrical stimulation.

The system 100 or similar systems may be used, for example, to carry out one or more aspects of any of the method 600 described herein. The system 100 or similar systems may also be used for other purposes.

It is to be understood that, while the electrodes 144 are discussed with respect to the system 100, such discussion is in no way limiting, and the electrodes 144 may be used in any other system and method not discussed herein. The electrodes 144 may also be fabricated in any context, and may be further used in any context, such as during a surgery or surgical procedure not involving a robot and/or robotic arm, during any medical procedure, during any non-medical procedure, etc.

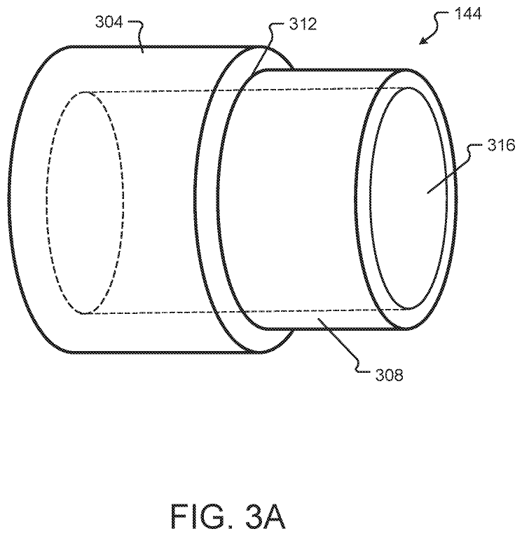

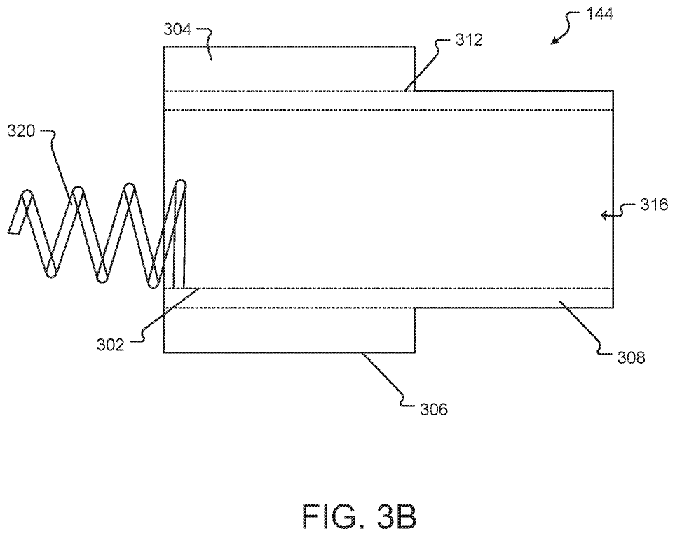

Turning to FIGS. 3A-3B, aspects of the electrodes 144 are shown in accordance with embodiments of the present disclosure. The electrode 144 may be fabricated as a bimetal ring or rod that includes an outer layer 304 (also referred to herein as an outer rod or an outer tube) and an inner layer 308 (also referred to herein as an inner rod or an inner tube). A diffusion layer 312 may be provided as an interface between the outer layer 304 and the inner layer 308. The diffusion layer 312 may form between the outer layer 304 and the inner layer 308 when the electrode 144 is fabricated, such as when the outer layer 304 and the inner layer 308 are diffusion bonded together. The electrode 144 is also depicted to include a hollow interior 316, although it may be possible to provide a solid inner layer 308 without a hollow interior 316. In some embodiments, coiled wiring 320 (e.g., a Ti-15Mo wire) may be inserted into the hollow interior 316 and welded to the inner layer 308. The wire may be welded to the hollow interior 316 through mechanical bonding, liquid phase welding (e.g., laser welding, resistance welding, etc.), combinations thereof, and the like. In some embodiments, the wiring 320 and/or the electrodes 144 may have a high fatigue performance, such as double the strain capability of MP35N®.

Both the outer layer 304 and the inner layer 308 may be or comprise metal or metal alloys, such that the electrode 144 can carry a first current between an inner surface 302 of the inner layer 308 and an outer surface 306 of the outer layer 304. In some embodiments, the electrode 144 may be a component of the surgical device 136, which may be or comprise an INS capable of being implanted into a patient. In such embodiments, the INS may be able to generate electrical pulses and pass the electrical pulses to the electrodes 144 through the wiring 320. The wiring 320 may be connected to the inner layer 308, such that the electrical pulse propagates through the wiring 320, into the inner layer 308, and out through the outer layer 304 and into proximate anatomical tissue. Such electrical pulses may be used for the purposes of stimulating the anatomical tissue to treat a patient's medical condition, to provide therapeutic relief or treatment to the patient, or for any other reason. In some embodiments, electrical signals that are generated in response to the electrical pulses (e.g., electrical signals generated from physiological and/or neurological responses from a patient's anatomical tissue) may be measured or recorded by the electrodes 144, and analyzed by the computing device 102, the surgical device 136, or the like.

One or more portions of the electrodes 144 may be radiopaque. In other words, the composition of the outer layer 304 and/or the inner layer 308 may cause the electrodes 144 to appear in fluoroscopic images, such as fluoroscopic images captured by the imaging devices 112. In some embodiments, the fluoroscopic images may be rendered to a display such that a user (e.g., a physician) can determine whether the electrodes 144 are disposed in the correct position when the electrodes 144, as part of an INS, are implanted into a patient.

The inner layer 308 may comprise Titanium or a Titanium alloy. In some embodiments, the inner layer 308 may comprise commercially pure Titanium (e.g., grade 1 Titanium, grade 2 Titanium, grade 3 Titanium, grade 4 Titanium, grade 7 Titanium, grade 11 Titanium, etc.). Alternatively or additionally, the inner layer 308 may be or comprise a Titanium alloy. For example, the inner layer 308 may be or comprise a Titanium alloy that includes Titanium and Tantalum, Niobium, Molybdenum, Zirconium, Tin, Vanadium, Tungsten, Iron, and/or Aluminum. The type of Titanium alloy is in no way limited to these types of alloys, however, and the type of Titanium or Titanium alloy used may vary depending on how the electrodes 144 are used, availability of materials, combinations thereof, and the like.

The outer layer 304 may comprise Platinum or a Platinum alloy. The Platinum in the inner layer 308 may offer an electrical charge density between the electrodes 144 and patient tissue. In other words, the use of Platinum may reduce the probability that the patient experiences an adverse reaction (e.g., an allergic reaction) to the electrodes 144 when electrodes 144 are implanted into patient tissue. In some embodiments, the outer layer 304 may comprise commercially pure Platinum (e.g., material whose composition is greater than 99% Platinum). Alternatively or additionally, the outer layer 304 may comprise a Platinum alloy, such as an alloy containing Platinum and Iridium (e.g., 90% Platinum, 10% Iridium; 70% Platinum, 30% Iridium; etc.).

As used herein and unless otherwise specified, the term “about” when referring to percentage values and compositions of one or more alloys, as well when referring to temperatures, pressures, or times, includes values with up to a 1% difference from the value specified. For example, a Platinum-Iridium alloy comprising about 90% Platinum and about 10% Iridium means that the Platinum-Iridium alloy can comprise 91% Platinum and 9% Iridium, or 89% Platinum and 11% Iridium, or any other percentage variation therebetween. As another example, about 30,000 psi includes pressures in the range from 29,700 psi to 30,300 psi. Alternatively, when so specified, “about” means that a 10% difference, a 5% difference, a 2% difference, or a 0.9% difference, or a 0.5% difference, or a 0.1% difference from the value specified is included. For the avoidance of doubt, each of the foregoing definitions of “about” can be used in connection with any embodiment described herein within the scope of the present disclosure. While various 1% difference examples are provided herein, it is to be understood that such examples (such as the examples of pressure ranges and/or alloy compositions) are not limiting, and additional or alternative pressure ranges and/or alloy compositions are possible.

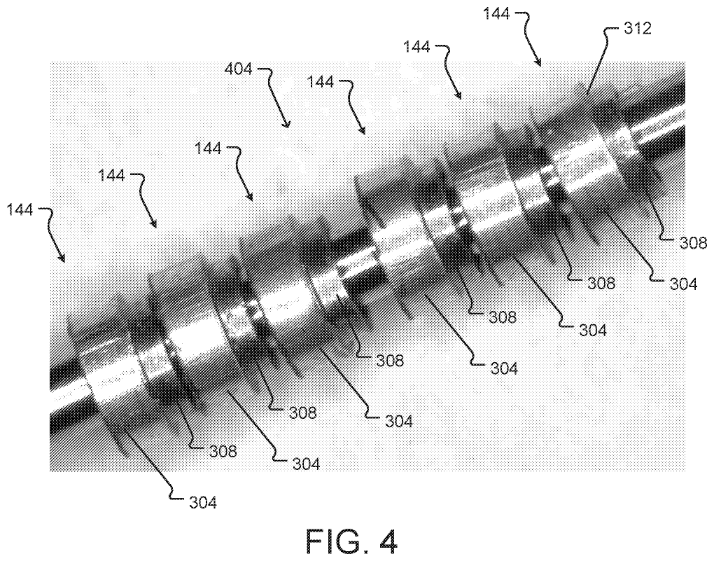

FIG. 4 depicts the fabrication of the electrodes 144 in accordance with embodiments of the present disclosure. The electrodes 144 may be fabricated from a composite tube 404 that is formed by extruding the outer layer 304 and the inner layer 308 in separate layers, and then diffusion bonding the two layers together. The composite tube 404 may be cut into sections, with each section capable of being used as an electrode 144.

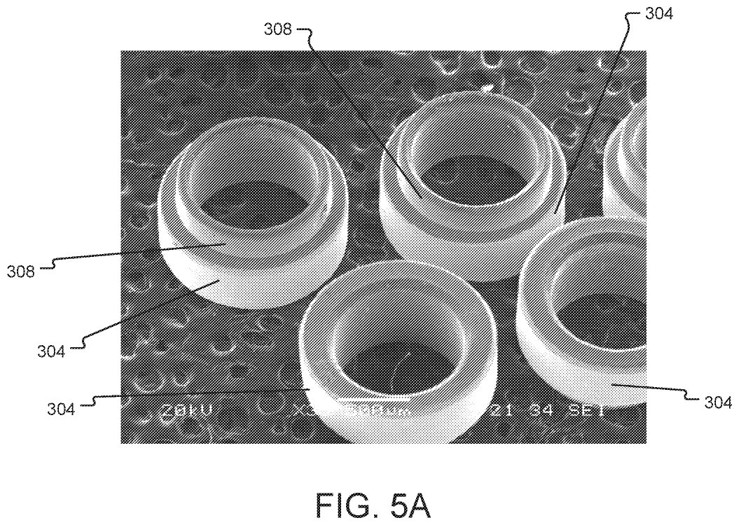

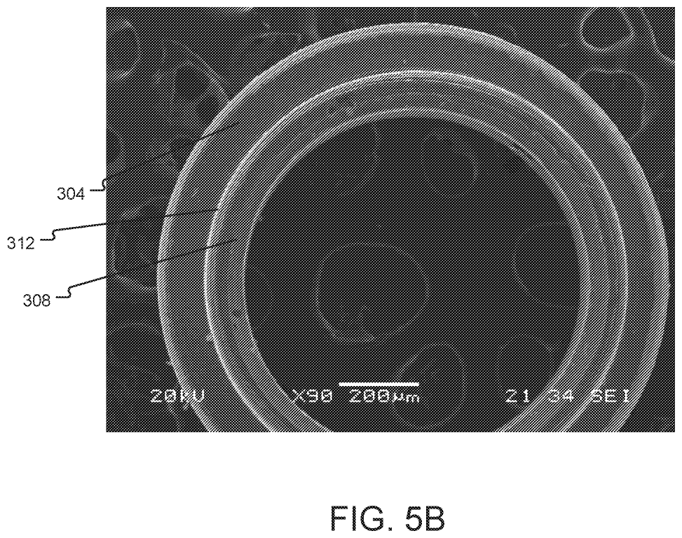

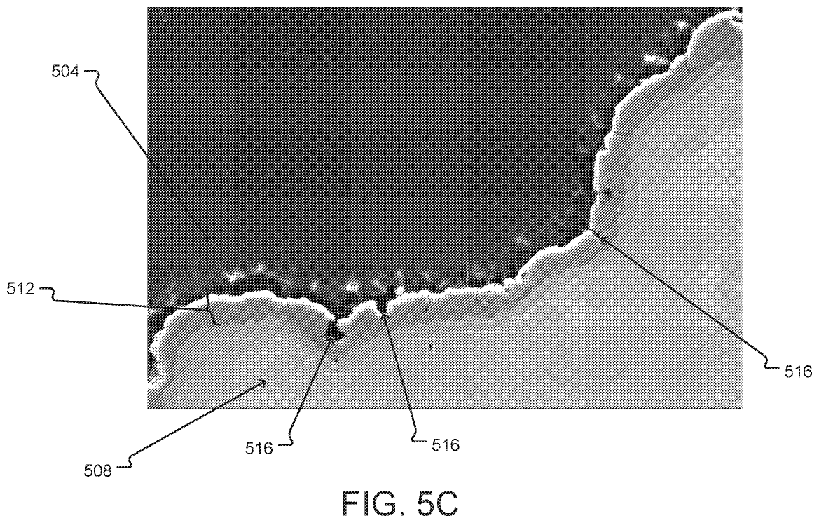

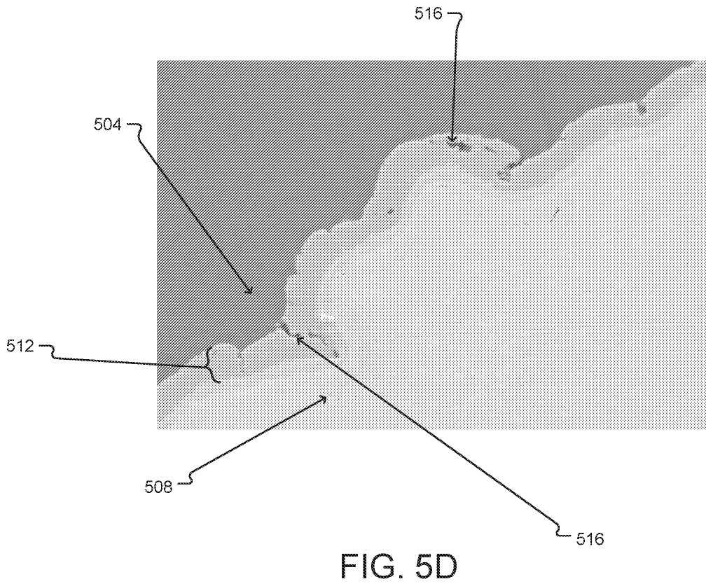

FIGS. 5A-5B illustrate images of the electrodes 144 in accordance with embodiments of the present disclosure. The images depict the bimetal rings that are capable of functioning as electrodes. The outer layer 304 and the inner layer 308 of the bimetal rings are diffusion bonded together, such that the diffusion layer 312 is formed between the outer layer 304 and the inner layer 308. FIG. 5C is an image of a diffusion layer 512 formed between Titanium or a Titanium alloy 504 and Platinum or platinum alloy 508 when the Titanium 504 and the Platinum 508 were diffusion bonded at 750 °C for 2 hours. As FIG. 5C shows, voids 516 appear in the diffusion layer 512, resulting in a weaker bond between the Titanium 504 and the Platinum 508. FIG. 5D shows the diffusion layer 512 formed between the Titanium 504 and the Platinum 508 formed after being diffusion bonded at 730 °C for 5 hours. At this temperature and time, the number of voids 516 appearing in the diffusion layer 512 is decreased, resulting in a stronger and thicker diffusion layer 512.

In some embodiments, the thickness of the diffusion layer may vary between about 0 micrometers (μm) and about 1.4 μm, and the grain size may range between about 20 μm and about 55 μm, based on diffusion bond processes (e.g., a diffusion bonding at 730 °C for various times between 0 minutes and 300 minutes, a diffusion bonding at 750 °C for various times between 0 minutes and 300 minutes, etc.). The thickness of the diffusion layer 512 may reflect the strength of the diffusion of the outer layer 304 and the inner layer 308, while grain size may be a measurement of the yield strength and tensile strength of the Titanium in the inner layer 308. In some embodiments, the grain size may be measured using a line and intersection count method.

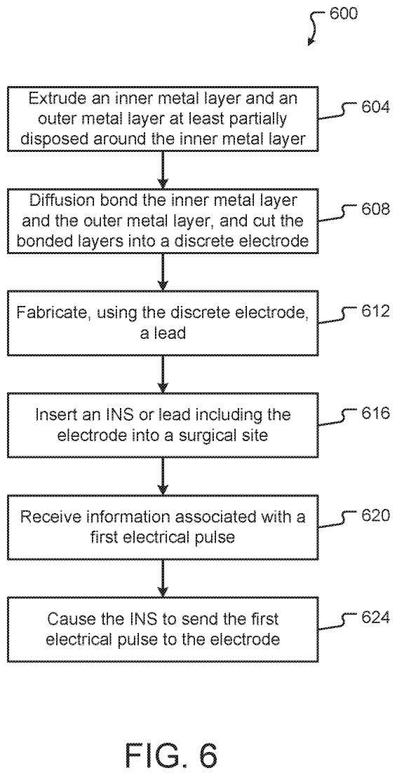

FIG. 6 depicts a method 600 that may be used, for example, to fabricate and use an electrode to stimulate patient tissue.

The method 600 comprises extruding an inner metal layer/rod and an outer metal layer/tube at least partially disposed around the inner metal layer/rod (step 604). The inner metal layer/rod may be similar to or the same as the inner layer 308, while the outer metal layer/tube may be similar to or the same as outer layer 304. In some embodiments, the inner metal layer may be or comprise Titanium or a Titanium alloy, while the outer metal layer may be or comprise Platinum or a Platinum alloy. The inner metal layer and the outer metal layer may both be extruded together to form a composite tube, with the outer metal layer surrounding the inner metal layer.

The method 600 also comprises diffusion bonding the inner metal layer and the outer metal layer, and cut the bonded layers into a discrete electrode (step 608). The composite tube formed from the inner metal layer and the outer metal layer may be diffusion bonded by placing the two layers in an oven or other heating device at high pressure and high temperature for a certain period of time. For example, the composite tube may be baked at 750 °C for 2 hours. In another example, the composite tube may be baked at 730 °C for 3 hours. While several examples are listed herein, the temperature and time is in no way limited to these examples, and alternative bake times at different temperatures are possible. For instance, the diffusion bonding may be performed at a temperature of between about 730 °C and about 750 °C for a time between about 175 minutes and about 300 minutes. More specifically, the diffusion bonding may be performed at a temperature between about 730 °C and about 738 °C for a time of between about 175 minutes and about 200 minutes. The diffusion bonding may occur in a high pressure environment, such as at 30,000 psi. While 30,000 psi is provided as an example, additional or alternative pressures or ranges of pressures may be used. In some embodiments, the diffusion bonding may occur in an inert environment or in the presence of an inert gas (e.g., argon). In such embodiments, the composite tube may be placed in the over, and the air in the oven may be evacuated and replaced by the inert gas before baking. Such an inert environment may prevent any one or more metals in the composite tube from reacting with the environment while undergoing the diffusion bonding process.

The diffusion bonding process may cause the inner metal layer and the outer metal layer to form a diffusion layer therebetween, bonding the two metal layers together. The composite tube may then be cut perpendicular to an extrusion direction into a discrete electrode. In some embodiments, the cuts may result in a plurality of discrete electrodes.

The method 600 also comprises fabricating, using the discrete electrode, a lead (step 612). The lead may include an insulated, current-carrying wire that extends from a proximal end that includes a plurality of connectors (e.g., connector rings) to a distal end that includes the discrete electrode. To fabricate the lead, the current-carrying wire (e.g., a Ti-15Mo wire) may be wrapped in an insulative layer (e.g., wrapped in plastic and/or rubber) to prevent the wire from inadvertently carrying current into an incorrect area of the patient. The proximal end of the wire may be connected to the INS or other surgical device using the plurality of connectors (e.g., connector rings), while the current-carrying wire may be welded to the inner metal layer of the electrode at the distal end of the lead. In some embodiments, multiple leads may be formed, each with a discrete electrode on the distal end thereof and each capable of being connected to the INS or other surgical device.

The method 600 also comprises inserting an INS or the lead including the electrode into a surgical site (step 616). Once the electrode has been cut from the composite tube, the electrode may be attached to leads or to an INS, and the lead or INS may be implanted into the surgical site. In one embodiment, the electrode may serve as a lead for treating OSA. In such an embodiment, the electrode may be implanted in or proximate the throat, such that the electrode can stimulate muscles in the throat to help treat OSA. In some embodiments, the electrode may be connected to another surgical device (e.g., surgical device 136) to receive instructions related to electrical signals. For example, the electrode may be wirelessly connected to the surgical device, with the surgical device causing the electrode to generate electrical impulses that stimulate the muscles of the throat.

The method 600 also comprises receiving information associated with a first electrical pulse (step 620). The received information may be from one or more sensors (e.g., sensors 148) that indicate that a first electrical pulse should be performed. For example, in the context of OSA, the one or more sensors may generate measurements related to patient heart rate, breathing rate, pulse, or the like. A processor (e.g., processor 104) may receive such measurements and determine that muscles in the throat should be stimulated to move the tongue forward to keep the airway clear while the patient is sleeping.

The method 600 also comprises causing the INS to send the first electrical pulse to the electrode (step 624). The processor may send one or more communications to the INS, which may send the first electrical pulse to the electrode, with the first electrical pulse stimulating the muscles. The electrode may generate a stimulation by carrying and/or amplifying the first electrical pulse into patient tissue, such as into patient tissue to stimulate the patient tissue.

The present disclosure encompasses embodiments of the method 600 that comprise more or fewer steps than those described above, and/or one or more steps that are different than the steps described above.

As noted above, the present disclosure encompasses methods with fewer than all of the steps identified in FIG. 6 (and the corresponding description of the method 600), as well as methods that include additional steps beyond those identified in FIG. 6 (and the corresponding description of the method 600). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An electrode, comprising:
an inner metal layer; and
an outer metal layer disposed at least partially around and diffusion bonded to the inner metal layer.

2. The electrode of claim 1, wherein the inner metal layer comprises Titanium or a Titanium alloy.

3. The electrode of claim 2, wherein the Titanium alloy comprises Titanium and one or more of Tantalum, Niobium, Molybdenum, Zirconium, Tin, Vanadium, Tungsten, Iron, and Aluminum.

4. The electrode of claim 1, wherein the inner metal layer is a Titanium-15Molybdenum alloy.

5. The electrode of claim 1, wherein the outer metal layer comprises Platinum or a Platinum alloy.

6. The electrode of claim 5, wherein the Platinum alloy comprises Platinum and Iridium.

7. The electrode of claim 1, wherein the diffusion bonding is performed at a temperature of between 730 degrees Celsius (C) and 750 °C and for a time between 175 minutes and 300 minutes.

8. The electrode of claim 7, wherein the diffusion bonding is performed at a temperature of between 730 °C and 738 °C and for a time of between 175 minutes and 200 minutes.

9. The electrode of claim 1, wherein the diffusion bonding occurs in a high pressure environment that includes an inert gas.

10. The electrode of claim 9, wherein the inert gas comprises argon, and wherein a pressure of the high pressure environment is about 30,000 pound-force per square inch (psi).

11. A method, comprising:
extruding an inner metal layer and an outer metal layer at least partially disposed around the inner metal layer; and
diffusion bonding the inner metal layer and the outer metal layer.

12. The method of claim 11, wherein the diffusion bonding is performed at a temperature of between 730 degrees Celsius (C) and 750 °C and for a time between 175 minutes and 300 minutes.

13. The method of claim 12, wherein the diffusion bonding is performed at a temperature of between 730 °C and 738 °C and for a time of between 175 minutes and 200 minutes.

14. The method of claim 11, wherein the inner metal layer comprises Titanium or a Titanium alloy.

15. The method of claim 14, wherein the Titanium alloy comprises Titanium and one or more of Tantalum, Niobium, Molybdenum, Zirconium, Tin, Vanadium, Tungsten, Iron, and Aluminum.

16. The method of claim 11, wherein the inner metal layer is a Titanium-15Molybdenum alloy.

17. The method of claim 11, wherein the outer metal layer comprises Platinum or a Platinum alloy.

18. The method of claim 17, wherein the Platinum alloy comprises Platinum and Iridium.

19. A system, comprising:
an implantable neural stimulator;
a lead with a proximal end connectable to the implant neural stimulator and a distal end that includes at least one electrode, the at least one electrode comprising:
an inner metal layer; and
an outer metal layer disposed at least partially around and diffusion bonded to the inner metal layer.

20. The system of claim 19, wherein the inner metal layer comprises Titanium or a Titanium alloy, and wherein the outer metal layer comprises Platinum or a Platinum alloy.

21. The system of claim 19, wherein the lead comprises a Titanium-15Molybdenum (Ti-15Mo) wire that connects the at least one electrode to connector rings attached to the proximal end of the lead.
